(19) United States
(12) Patent Application Publication
(10) Pub. No.: US 2007/0054978 A1
(43) Pub. Date: Mar. 8, 2007

(54) One-liquid medical and dental curable composition

(75) Inventors: Kiyomi Futigami, Kyoto (JP); Kunio Ikemura, Kyoto (JP); Kensuke Ichizawa, Kyoto (JP)

Correspondence Address: STEVENS DAVIS MILLER & MOSHER, LLP, 1615 L STREET, NW, SUITE 850, WASHINGTON, DC, 20036, US

(73) Assignee: KABUSHIKI KAISHA SHOFU, Kyoto-shi (JP)

(21) Appl. No.: 11/220,377

(22) Filed: Sep. 7, 2005

(51) Int. Cl. A61K 6/08

(52) U.S. Cl. 523/116; 523/205

(57) ABSTRACT

The present invention provides a one-liquid curable composition comprising micro- and/or nano-capsules. More specifically, the present invention provides a one-liquid curable composition characterized in that a substance encapsulated within each micro- and/or nano-capsule and a substance enveloping said micro- and/or nano-capsule contain a combination of more than one material that can coexist with one another, selected arbitrary from radically-polymerizable monomers, polymerization initiators, polymerization accelerators, electrolyte polymers, electrolyte polymer solutions, hydroxycarboxylic acids, hydroxycarboxylic acid solutions, acid-reactive fillers, and acid-inactive fillers, and also characterized in that a wall material of said micro- and/or nano-capsule is an organic compound, and/or an inorganic compound, and/or an organic/inorganic complex compound.

### FIELD OF THE INVENTION

The present invention relates to a one-liquid curable composition comprising micro- and/or nano-capsules.

### BACKGROUND OF THE INVENTION

Unlike the case of the general industrial field, materials designed in the field of dentistry are required to have material characteristics that provide good physical properties and mechanical and lasting adhesion, capable of hardening within a few minutes under intraoral conditions involving complete infiltration with saliva, breath or dental pulp fluid, a high acid level induced by bacterial metabolism, protein buildup, rapid temperature change caused by dietary intake, etc., and capable of enduring constant occlusal force applied after being cured. Among these properties, material-handling properties in particular are crucially important because they are a starting point of cumbersome dental treatment and they influence on whether or not material characteristics are fully achieved intraorally. Handling properties are deeply related to packaging techniques and product forms.

Due to an advantage of no mixing step and shorter treatment time, clinicians commonly use one-liquid photopolymerizable composite resins and bonding agents for restorative treatment and one-liquid photopolymerizable composite resins for crowns and bridges. Conventional polymerization initiators that have been disclosed to date include alpha-diketone such as D,L-camphorquinone and N,N-dimethylamino ethyl methacrylate as in Kokoku (Japanese examined patent publication) No. 54-10986, and alpha-diketone and N,N-dimethylamino benzoate ethyl ester as in Kokoku (Japanese examined patent publication) No. 60-26002, both of which are effective for visual light polymerization that goes through an exciplex, enabled by alpha-diketone excited by exposure to light and a photoreducing agent used as a hydrogen donator. With such polymerization initiators, however, light-curing properties decrease when adhesive monomers having acid groups coexist, or when a concentration of these initiators is particularly low.

Meanwhile, adhesive resin cements, which contain adhesive monomers having acid groups such as a phosphate ester group or a carboxyl group within a molecule, and have a basic configuration of a redox polymerization initiating system consisting of benzoyl peroxide and aromatic tertiary amine, are highly evaluated for their excellent adhesion to enamel/dentin, metal or ceramics, and thus widely used for dental prosthetic purposes. However, these resin cements have some drawbacks. Their curing properties and bonding properties are reduced by a charge-transfer complex formed when an add group within an adhesive monomer comes in contact with aromatic tertiary amine. Also their fluoride releasing capability is markedly lower than that of after-mentioned (resin modified) glass ionomer cements.

Since the invention by Wilson and Kent in 1973 (British Patent No. 1,316,129), glass ionomer (glass polyalkenoate) cements have been used to date on a day-to-day basis in the field of clinical dentistry. Clinical expectations towards these cements are high because of their high bio-compatibility, favorable adhesiveness to natural teeth, and excellent fluoride releasing capability that has anticarious potential. The important aspect in said cements is to have a reactive layer of glass ionomer comprised of hydrogel salts formed by a reaction between a basic calcium aluminofluorosilicate glass and an acidic polyelectrolyte (polyalkenoic acid), i.e. a homopolymer or a copolymer of unsaturated carboxylic acid. This aspect is clinically acclaimed for its high-level fluoride release and recharge capability. However, there are some problems with said cements, which are that they require time to obtain sufficient hardness for polishing after a restoration procedure due to slowness in curing reaction thereof between glass and polyalkenoic acid, and that the cured surface of glass ionomer is sensitive to oral moisture, which leads to degradation.

Recently, resin-containing glass ionomer cements, called resin modified glass ionomer cements have started to gain popularity. Japanese Patent Publication No.2869078 discloses the effect of grafted polyalkenoic acid having an unsaturated double-bond group introduced into its side chain. These cements involve a chemical- or photo-polymerization reaction of the resin, which accelerates the curing speed. They also show improvement in the sensitivity to oral moisture. With the powder-liquid type of such cements, however, powder and liquid must be precisely dispensed so as to maintain the physical properties. In other words, measuring errors cause lifting or dislodgment of metal crowns or inlays at the time of bonding. In an attempt to solve such a problem above, capsule-type glass ionomer cements have been developed. In this type of glass ionomer cements, pre-measured powder and liquid are separated by a membrane within a capsule and are mixed by the use of mechanical vibration such as a mixer. A problem yet remains as the cement within a capsule starts curing immediately after the components are mixed.

Kokai (Japanese unexamined patent publication) No. 11-228327 discloses a technology relating to a paste & paste type resin modified glass ionomer cement. This type of cement has solved the problem of measuring errors by controlling a powder/liquid ratio at the time of manufacturing, and has improved handling properties and the problem with sensitivity to moisture

As described above, acid-base reactive-type glass ionomer cements and redox polymerization-type resin cements still remain to require the mixing step since they are supplied separately in two or more packages. Therefore, hardened cement is prone to inclusion of air bubbles, resulting in insufficient physical strength. In order to overcome this problem, a one-pack light-curable dental cement containing a pre-formed glass ionomer filler was proposed, as described in Japanese Patent No. 3497508 or U.S. Pat. No. 5,883,153. This pre-formed glass ionomer filler is produced by pre-reacting acid-reactive glass and polyalkenoic acid to form a hydrogel salt, which is then freeze-dried to obtain a xero-gel, which is finally pulverized. This one-paste composition is well received as a new category of dental restorative materials called “Giomer,” characterized by its excellent physical properties achieved by cutting out the need of the mixing step which means no air bubbles introduced in the resulting hardened material, and its capability to release the appropriate amount of fluoride. Compared to conventional (resin modified) glass ionomer cements, this newly-categorized material, however, exhibits less fluoride release, and is lower in self-adhesiveness due to consumed polycarboxylic acid.

As described above, since the introduction of the photo polymerization technology into the field of clinical dentistry, one-paste type dental composite resins have been widely diffused owing to the packaging convenience of one-liquid photo polymerization initiator systems. On the other hand, glass ionomer type materials are not exactly in widespread use, despite of their noticeable fluoride-releasing capability and high level of biosafety. This is because the one-liquid packaging technology has not yet been established. More specifically, currently-available glass ionomers and resin cements used in the field of clinical dentistry, whether they are a paste/liquid type or paste/paste type, require a mixing step, which results in the existence of air bubbles introduced into a hardened material and insufficient physical properties. Any fundamental solutions to these problems have not yet been found. Since these materials are essential in the field of conservative dentistry and prosthodontic practice, development of one-liquid or one-paste glass ionomer cements or resin cements that involve simple and effective operation has been eagerly anticipated. No conventional technology has yet proposed a method wherein acid-base reactive components and redox polymerization initiators consisting of benzoyl peroxide and amines can coexist within a single paste or a single liquid. In other words, not a single invention has been developed relating to one-liquid or one-paste glass ionomers or resin cements that overcome the existing fundamental problems, wherein their components can coexist within a single paste or a single liquid, and wherein no mixing step is essentially required.

### SUMMARY OF THE INVENTION

Particularly in the dental field, there has not existed a one-liquid curable composition comprising micro- and/or nano-capsules.

It is conventionally impossible to have reactive components coexist with one another within a single-liquid composition without keeping them apart. Such components include acid-base reactive components where polyalkenoic acid reacts with acid-reactive glass to form a glass ionomer, and redox polymerization initiators consisting of benzoyl peroxide and aromatic tertiary amine. Thus there has not been a one-paste or one-liquid curable composition containing such reactive components. In other words, there has not existed a one-liquid curable composition characterized in that a substance encapsulated within each micro- and/or nano-capsule and a substance enveloping said micro- and/or nano-capsule contain a combination of more than one material that can coexist with one another, selected arbitrary from radically-polymerizable monomers, polymerization initiators, polymerization accelerators, electrolyte polymers, electrolyte polymer solutions, hydroxycarboxylic acids, hydroxycarboxylic acid solutions, acid-reactive fillers, and acid-inactive fillers, and also characterized in that a wall material of said micro- and/or nano-capsule is an organic compound, and/or an inorganic compound, and/or an organic/inorganic complex compound.

Moreover, there has not been provided a one-liquid curable composition wherein its components such as acid-base reactive components and redox polymerization initiators consisting of benzoyl peroxide and aromatic tertiary amine, which are conventionally not able to coexist within a single paste or a single liquid, are micro- and/or nano-encapsulated in order to have them coexist within a single paste or a single liquid, and wherein a mixing operation is not essentially required, i.e. a polymerization reaction can be initiated whenever needed by simply giving external energy to break said capsules, thereby providing easy and satisfactory operability and excellent physical properties resulting from the absence of air bubbles, which are conventionally incorporated during a mixing operation.

The present invention relates to a one-liquid curable composition characterized in that a substance encapsulated within each micro- and/or nano-capsule and a substance enveloping said micro- and/or nano-capsule contain a combination of more than one material that can coexist with one another, selected arbitrarily from radically-polymerizable monomers, polymerization initiators, polymerization accelerators, electrolyte polymers, electrolyte polymer solutions, hydroxycarboxylic acids, hydroxycarboxylic acid solutions, acid-reactive fillers, and acid-inactive fillers, and also characterized in that a wall material of said micro- and/or nano-capsule is an organic compound, and/or an inorganic compound, and/or an organic/inorganic complex compound. In the conventional technologies, it was especially impossible to have, for example, acid-base reactive components wherein polyalkenoic acid reacts with acid-reactive glass to form a glass ionomer, or redox polymerization initiators consisting of benzoyl peroxide and aromatic tertiary amine, coexist within a single paste or a single liquid. The present invention relates to a one-liquid curable composition wherein such reactive components that are not able to coexist with one another are micro- and/or nano-encapsulated in order to have them coexist within a single paste or a single liquid, and wherein a mixing operation is not essentially required, i.e. a polymerization reaction can be initiated whenever needed by simply giving external energy to break said capsules, thereby providing easy and satisfactory operability and excellent physical property resulting from the absence of air bubbles, which are conventionally incorporated during a mixing operation.

The present invention is a one-liquid curable composition consisting of radically-polymerizable monomers with acid groups and radically-polymerizable monomers without acid groups.

The present invention is a one-liquid curable composition wherein a polymerization initiator thereof is a redox chemical polymerization agent

The present invention is a one-liquid curable composition wherein an electrolyte polymer and an electrolyte polymer solution thereof are polyalkenoic acid and its solution.

The present invention is a one-liquid curable composition wherein hydroxycarboxylic acid and a hydroxycarboxylic acid solution thereof are a compound and its solution having hydroxyl groups within a molecule, such as tartaric acid and malic acid.

The present invention is a one-liquid curable composition wherein an acid-reactive filler thereof is of basic glass powder.

The present invention is a one-liquid curable composition wherein an acid-inactive filler thereof is an inorganic filler and an organic complex filler.

The present invention is a one-liquid curable composition wherein an organic compound, an inorganic compound, and an organic/inorganic complex compound used for a wall material of micro- and/or nano-capsules are an organic polymer compound, an inorganic polymer compound, and a composite of an organic polymer compound and organic filler, respectively.

The present invention is a one-liquid curable composition wherein a flux and/or a polymerization inhibitor can be added.

The present invention is a one-liquid curable composition wherein a polymerization reaction can be initiated by simply giving external energy extraorally to break said capsules.

The present invention is a one-liquid curable composition characterized by containing micro- and/or nanocapsules, and characterized in that a substance encapsulated within each micro- and/or nano-capsule and a substance enveloping said micro- and/or nano-capsule contain a combination of more than one material that can coexist with one another, selected arbitrarily from radically-polymerizable monomers, polymerization initiators, polymerization accelerators, electrolyte polymers, electrolyte polymer solutions, hydroxycarboxylic acids, hydroxycarboxylic acid solutions, acid-reactive fillers, and acid-inactive fillers, and also characterized in that a wall material of said micro- and/or nano-capsule is an organic compound and/or an inorganic compound, and/or an organic/inorganic complex compound. Especially in the conventional technologies, components such as acid-base components and redox polymerization initiators consisting of benzoyl peroxide and aromatic tertiary amine were not able to coexist within a single paste or a single liquid. We successfully achieved to have these reactive components exist stably within a single paste or a single liquid by having them micro- and/or nano-encapsulated, and we also discovered a method to initiate a polymerization reaction whenever needed by simply giving external energy extraorally to break said capsules, providing excellent handling properties and curing ability to said one-liquid curable composition because no mixing operation is essentially required that means no air bubbles are essentially involved Such achievement and discovery led to the present invention.

### DETAILED DESCRIPTION OF THE PREFERRED EMBODIMENT

In the implementation of a one-liquid curable composition of the present invention, there is an advantage of markedly improved handling properties for reactive substances that are conventionally kept in more than one separate package because they can be provided as a one-paste or a one-liquid type by means of the encapsulation technology of the present invention.

A method to polymerize a one-paste or one-liquid curable composition containing micro- and/or nano-capsules of the present invention is cured as a result of external energy can be arbitrarily selected from conventional methods where substances separately kept in more than one package are mixed together to initiate a reaction at the point of use. More specifically, a curing method can be arbitrarily selected from among, for example, an acid-base reaction, a chemical polymerization, a dual cure method, a photo polymerization, an additive polymerization, a ring-opening polymerization, a polycondensation method, etc., depending on a mode to be employed. The best specific mode for carrying out the invention especially includes a one-paste dental cement and a one-liquid dental adhesive, characterized by marked improvement in handling properties of one-paste glass ionomer cements and on-paste resin cement, and characteristics and adhesiveness of cured materials.

As some modes of use, a one-liquid curable composition of the present invention can be used for one-paste glass ionomer cements and one-paste resin modified glass ionomer cements. It is also used not only in the field of dentistry, but also in the medical fields, especially in the field of orthopedic surgery involving bone cements, due to its excellent curing ability achieved by the markedly improved handling properties and the essential absence of air bubbles because of no mixing process involved.

The present invention of a one-liquid curable composition is characterized by containing micro- and/or nano-capsules, and can be carried out in the intended mode where a substance encapsulated within said capsule and a substance in combination of said capsule comprising said one-liquid curable composition is a single material and/or a combination of materials that are able to coexist with one another, selected arbitrarily from radically-polymerizable monomers, polymerization initiators, polymerization accelerators, electrolyte polymers, electrolyte polymer solutions, hydroxycarboxylic acids, hydroxycarboxylic acid solutions, acid-reactive fillers, and acid-inactive fillers.

Said radically-polymerizable monomers can be used alone, or two or more types of the compounds can be used in combination. A blending proportion of the foregoing radically-polymerizable monomers varies widely according to each mode for carrying out the present invention, from 1 to 99wt % for the total quantity of the one-liquid curable composition of the present invention, preferably from 30 to 90wt %, and more preferably from 40 to 70wt %.

If the proportion is less than 1 or more than 99wt %, the curing ability of the one-liquid curable composition becomes insufficient, and its adhesiveness and physical properties tend to be deteriorated.

Polymerization initiators to be used for the one-liquid curable composition of the present invention can be selected from polymerization initiators that are used in dentistry and the general industrial field. Their blending proportion is 0.01-15 wt % for the total quantity of the one-liquid curable composition of the present invention, preferably 0.1-10 wt %, and more preferably 0.5-5 wt %.

Polymerization catalysts contained in a one-liquid curable composition of the present invention include, for example, ascorbic acids, and barbituric acids to be hereinafter described.

These radical polymerization initiators and water-soluble polymerization initiators can be used alone, or two or more types of these compounds can be used in combination. A blending proportion of these polymerization initiators is 0.01-10 wt % for the total quantity of the radically-polymerizable monomers in the one-liquid curable composition of the present invention, preferably 0.05-5 wt %, and more preferably 0.1-3 wt %.

Polymerization accelerators in the curable composition of the present invention used in combination with a polymerization initiator can be selected from compounds that are conventionally used as photo polymerization accelerators or autopolymerization accelerators. Particularly preferred are barbituric acid derivatives, aromatic amines, aliphatic amines, and tin compounds.

These polymerization accelerators can be used alone, or two or more types of these compounds can be used in combination. A blending proportion of these polymerization accelerators is 0.01-15 wt % for the total quantity of the one-liquid curable composition of the present invention, preferably 0.05-5 wt %, and more preferably 0.1-3 wt %.

Said acid-reactive fillers and acid-inactive fillers can be used alone, or two or more types of the compounds can be used in combination. A blending proportion of these fillers is respectively 0.1-70 wt % for the total quantity of the one-liquid curable composition of the present invention, preferably 1-60 wt %, and more preferably 3-50 wt %.

A one-liquid curable composition of the present invention achieves an initial aim, according to the mode for carrying out the invention, by using an external force to break a wall material of micro- and/or nano-capsules to release an encapsulated substance from protection and mix with materials comprising the one-liquid curable composition, which causes curing of the mixture. While not particularly limited as long as it is a force that is required to break a wall material of the capsules, and that generates medically safe energy, the external force to be applied can be one or more forces selected from electromagnetic waves such as visible light, near-ultraviolet rays, ultraviolet rays, and x-rays, a magnetic field, ultrasound, pressure such as occlusal stress, heat, agitation by reciprocating oscillation, and the like.

More specifically, the present invention of a one-liquid curable composition can be carried out in a way where one or both of reactive components such as materials capable of causing an acid-base reaction or a redox reaction, or materials capable of generating radicals upon contact with one another are preliminarily microencapsulated, which enables a radically-polymerizable monomer, a photo polymerization initiator, a filler, an ultrafine particle filler and a polymerization inhibitor to be mixed together to form a paste that is filled in a mini plastic syringe and supplied as a curable composition such as a one-paste light-curable resin modified glass ionomer cement, a one-paste light-curable resin cement, or a one-liquid light-curable bonding agent, and the outer wall material of the microcapsules contained in the composition is broken by means of ultrasound prior to use to initiate a reaction, and during the reaction the composition is applied onto a site to be treated in a patient.

### EXAMPLES

Next, the invention will be described with the following working and comparative examples hereinbelow. It must be noted that these examples do not limit the invention.

### Example 1

### Microencapsulation of Polyacrylic Acid Solution

Materials used were glass ionomer cement CX-Plus liquid (manufactured by Shofu, Inc.) as a core material (encapsulated substance); condensed hexaglycerin recinoleate (abbreviated as Sunsoft 818SX: manufactured by Taiyo Kagaku Co., Ltd.) as a surfactant that forms an organic phase, toluene as an organic solvent; methylmethacrylate (MMA) as a monomer; benzoyl peroxide (BPO: manufactured by Nacalai Tesque, Inc.) as a polymerization initiator; ion-exchanged water as outer phase water; and gelatin (manufactured by Nacalai Tesque, Inc.) as a dispersion stabilizer for outer phase water.

Firstly, 10 g of MMA containing 2 wt % of BPO were dissolved in 50 mL of toluene containing 0.60 g of condensed hexaglycerin recinoleate. Then, 25 mL of CX-Plus liquid, i.e. an inner water phase of the core material, was stirred into the resultant organic phase for 10 minutes using a Physcotron homogenizer at 5000 rpm under cooling at 10° C. to obtain a primary dispersion phase. Next, gelatin was stirred and dissolved into 300 mL of ion-exchanged water at 60° C. to make 3 wt %, so as to prepare an outer water phase. Then, while keeping the temperature of the outer water phase at 40° C., the primary dispersion phase was added using a three-one motor agitator at 500 rpm to obtain a combined emulsion. While rotated, the combined emulsion was heated up and kept at 80° C. for 6 hours. The emulsion was then desolvated for approximately 2 hours with its reaction system kept under slightly-reduced pressure. After completion of the reaction, obtained microcapsules were separated by filtration under reduced pressure, then washed with copious ion-exchanged water, and dried with hot air at 40° C., thereby obtaining microcapsules enclosing pale yellow polyacrylic acid solution. The yield was 89%. The average capsule diameter analyzed was approximately 200 μm. It was also observed that the structure of the capsules was relatively mononucleated.

### Examples 2-9

### Manufacturing of Microcapsules Containing a Polyacrylic Acid Solution

Test reagents used were the same as in Example 1. Conditions such as concentrations and agitation rates employed varied as shown in Table 1. The amount of toluene used as an organic solvent was fixed to 50 mL.

TABLE 1
Conditions for microencapsulation of polyacrylic acid solution

| | | | | | | Agitation | Agitation |
|---|---|---|---|---|---|---|---|
| | | | | | | rate for | rate for |
| | CX-Plus | | Sunsoft | Ion-exchanged | | primary | secondary |
| Example | Liquid | MMA | 818SX | water | Gelatin | dispersion | dispersion |
| No. | (mL) | (g) | (g) | (mL) | (wt %) | (rpm) | (rpm) |
| 1 | 25 | 10 | 0.60 | 300 | 3 | 5000 | 500 |
| 2 | 25 | 10 | 0.60 | 300 | 4 | 5000 | 500 |
| 3 | 25 | 10 | 0.60 | 300 | 5 | 5000 | 500 |
| 4 | 30 | 10 | 0.60 | 360 | 3 | 5000 | 500 |
| 5 | 35 | 9 | 0.59 | 420 | 3 | 5000 | 500 |
| 6 | 40 | 8 | 0.58 | 480 | 3 | 5000 | 500 |
| 7 | 25 | 10 | 0.60 | 300 | 3 | 10000 | 500 |
| 8 | 25 | 10 | 0.60 | 300 | 3 | 15000 | 750 |
| 9 | 25 | 10 | 0.60 | 300 | 3 | 20000 | 1000 |

TABLE 2
Results for microencapsulation of polyacrylic acid solution

| | | Average | |
|---|---|---|---|
| | | capsule | |
| Example | | diameter | |
| No. | Yield (%) | (μm) | Form |
| 1 | 89 | 200 | Mononucleated capsules |
| 2 | 90 | 170 | Mononucleated capsules |
| 3 | 92 | 150 | Mononucleated capsules |
| 4 | 93 | 200 | Mononucleated capsules |
| 5 | 95 | 150 | Combination of mononucleated and |
| | | | multinucleated capsules |
| 6 | 91 | 120 | Combination of mononucleated |
| | | | and multinucleated capsules |
| 7 | 85 | 100 | Multinucleated capsules |
| 8 | 83 | 70 | Multinucleated capsules |
| 9 | 75 | 50 | Multinucleated capsules |

### Example 10

### Microencapsulation of Tartaric Acid Solution

Materials used were tartaric acid solutions with various concentrations as core materials; Noigen ET-83 (abbreviated as ET-83: manufactured by Dai-ichi Kogyo Seiyaku Co., Ltd.) as a surfactant that forms an organic phase; triethylene glycol dimethacrylate (TEGDMA: manufactured by Shin-Nakamura Chemical Co., Ltd.) as a monomer; azoisobutyronitrile (AIBN) as a polymerization initiator; ion-exchanged water as outer phase water; and polyvinyl alcohol with a polymerization degree of 3500 and a saponification value of 86.0-90.0 mol % (PVA: manufactured by Wako Pure Chemical Industries, Ltd.) as a dispersion stabilizer for outer phase water.

Firstly, 1.50 g of ET-83 were dissolved in 50 mL of TEGDMA. Then, 25 mL of a 100 wt % tartaric acid solution, i.e. an inner water phase of the core material, was stirred into the resultant organic phase for 10 minutes using a Physcotron homogenizer at 5000 rpm under cooling at 10° C. to obtain a primary dispersion phase. Next, PVA was stirred and dissolved into 300 mL of ion-exchanged water at 70° C. to make 5 wt %, so as to prepare an outer water phase. Then, while keeping the temperature of the outer water phase at 40° C. the primary dispersion phase was added using a three-one motor agitator at 500 rpm to obtain a combined emulsion. While rotated, the combined emulsion was heated up and kept at 80° C. for 6 hours. After completion of the polymerization reaction, obtained microcapsules were separated by filtration under reduced pressure, then washed with copious ion-exchanged water, and dried with hot air at 40° C., thereby obtaining microcapsules enclosing white tartaric acid solution. The yield was 92%. The average capsule diameter analyzed was approximately 100 μm. The SEM observation showed that the structure of the capsules was relatively mononucleated.

### Examples 11-18

### Microencapsulation of a Tararic Acid Solution

Test reagents used were the same as in Example 10. Conditions such as concentrations and agitation rates employed varied as shown in Table 3.

TABLE 3
Conditions for microencapsulation of tartaric acid solution

| | | | | | | Agitation | Agitation |
|---|---|---|---|---|---|---|---|
| | | | Tartaric | Tartaric | | rate for | rate for |
| | | | acid | acid | | primary | secondary |
| Example | ET-83 | TEGDMA | concentration | solution | PVA | dispersion | dispersion |
| No. | (g) | (mL) | (wt %) | (mL) | (wt %/mL) | (rpm) | (rpm) |
| 11 | 1.50 | 50 | 15 | 25 | 5/300 | 5000 | 500 |
| 12 | 1.50 | 50 | 20 | 25 | 5/300 | 5000 | 500 |
| 13 | 0.60 | 20 | 10 | 25 | 5/120 | 5000 | 500 |
| 14 | 0.90 | 30 | 10 | 25 | 5/180 | 5000 | 500 |
| 15 | 1.20 | 40 | 10 | 25 | 5/240 | 5000 | 500 |
| 16 | 1.50 | 50 | 10 | 25 | 1/300 | 5000 | 500 |
| 17 | 1.50 | 50 | 10 | 25 | 3/300 | 5000 | 500 |
| 18 | 1.50 | 50 | 10 | 25 | 5/300 | 20000 | 1000 |

TABLE 4
Results for microencapsulation of tartaric acid solution

| | | Average | |
|---|---|---|---|
| | | capsule | |
| | Yield | diameter | |
| Example | (%) | (μm) | Form |
| 11 | 90 | 110 | Mononucleated capsules |
| 12 | 92 | 130 | Mononucleated capsules |
| 13 | 93 | 200 | Mononucleated capsules |
| 14 | 95 | 160 | Combination of mononucleated and |
| | | | multinucleated capsules |
| 15 | 91 | 100 | Combination of mononucleated and |
| | | | multinucleated capsules |
| 16 | 85 | 200 | Combination of mononucleated and |
| | | | multinucleated capsules |
| 17 | 83 | 120 | Combination of mononucleated and |
| | | | multinucleated capsules |
| 18 | 75 | 70 | Multinucleated capsules |

### Examples 19-27

### Microencapsulation of Hydroxycarboxylic Acid and Polyacrylic Acid Solutions

Basic procedures were the same as in Example 10. Conditions such as types/concentrations of monomers and hydroxycarboxylic acids varied as shown in Table 5.

TABLE 5
Conditions for microencapsulation of hydroxycarboxylic acid and
polyacrylic acid solutions

| | | Amount | Type and amount (g) of |
|---|---|---|---|
| | | of | hydroxycarboxylic acid |
| Example | Type of monomers/ | Monomer | Amount of |
| No. | weight ratio(wt %) | (mL) | CX-Plus liquid (mL) |
| 19 | EGDMA | 50 | Tartaric acid (1.0) |
| | | | CX-Plus liquid (25) |
| 20 | EGDMA | 50 | Tartaric acid (2.0) |
| | | | CX-Plus liquid (25) |
| 21 | Bis-GMA 60 wt % | 50 | Tartaric acid (2.0) |
| | EGDMA 40 wt % | | CX-Plus liquid (25) |
| 22 | Bis-GMA 60 wt % | 50 | Malic acid (2.0) |
| | EGDMA 40 wt % | | CX-Plus liquid (25) |
| 23 | Bis-GMA 60 wt % | 50 | Tartaric acid (2.0) |
| | EGDMA 40 wt % | | CX-Plus liquid (25) |
| 24 | Bis-GMA 60 wt % | 50 | Malic acid (2.5) |
| | TEGDMA 40 wt % | | CX-Plus liquid (25) |
| 25 | MMA 70 wt % | 50 | Tartaric acid (3.2) |
| | Bis-GMA 30 wt % | | CX-Plus liquid (40) |
| 26 | MMA 70 wt % | 50 | Malic acid (3.2) |
| | Bis-GMA 30 wt % | | CX-Plus liquid (40) |
| 27 | MMA 70 wt % | 40 | Tartaric acid (3.2) |
| | Bis-GMA 30 wt % | | CX-Plus liquid (40) |

Note:
The amount of the surfactant (ET-83) was fixed to 1.50 g.
Hydroxycarboxylic acids were dissolved in CX-Plus liquid.
The outer phase water used was fixed to 5 wt % - 300 mL of polyvinyl alcohol (PVA: manufactured by Wako Pure Chemical Industries, Ltd.) with a polymerization degree of 3500 and a saponification value of 86.0-90.0 mol %.
Agitation rates for the primary and secondary dispersions were the same as in Example 10.
EGDMA: ethylene glycol dimethacrylate
Bis-GMA: bisphenol A-diglycidyl methacrylate

TABLE 6
Results for microencapsulation of hydroxycarboxylic acid and polyacrylic
acid solutions

| | | Average | |
|---|---|---|---|
| | | capsule | |
| Example | Yield | diameter | |
| No. | (%) | (μm) | Form |
| 19 | 91 | 70 | Mononucleated capsules |
| 20 | 85 | 80 | Mononucleated capsules |
| 21 | 88 | 150 | Mononucleated capsules |
| 22 | 90 | 170 | Combination of mononucleated and |
| | | | multinucleated capsules |
| 23 | 89 | 160 | Combination of mononucleated and |
| | | | multinucleated capsules |
| 24 | 92 | 190 | Combination of mononucleated and |
| | | | multinucleated capsules |
| 25 | 95 | 70 | Combination of mononucleated and |
| | | | multinucleated capsules |
| 26 | 96 | 65 | Multinucleated capsules |
| 27 | 93 | 80 | Multinucleated capsules |

### Example 28

### Synthesis of Capsules Having an Inorganic Capsule Wall

Materials used were ion-exchanged water as a core material; Noigen ET-83 (hereinafter referred to as ET-83: manufactured by Dai-ichi Kogyo Seiyaku Co., Ltd.) as a surfactant that forms an organic phase; orthotetraethyl silicate (TEOS: manufactured by Nacalai Tesque, Inc.) as a monomer; 0.1M hydrogen chloride solution (HCl) as a polymerization initiator, ion-exchanged water as outer phase water, and polyvinyl alcohol (PVA) with a polymerization degree of 3500 and a saponification value of 86.0-90.0 mol % as a dispersion stabilizer for outer phase water.

Firstly, 1.50 g of ET-83 was dissolved in 50 mL of TEOS. Then, 25 mL of ion-exchanged water, i.e. an inner water phase of the core material, was stirred into the resultant organic phase for 10 minutes using a Physcotron homogenizer at 5000 rpm under cooling at 10° C. to obtain a primary dispersion phase. Next, PVA was stirred and dissolved into 300 mL of ion-exchanged water at 70° C. to make 5 wt %, so as to prepare an outer water phase. Then, while keeping the temperature of the outer water phase at 40° C., the primary dispersion phase was added using a three-one motor agitator at 500 rpm to obtain a combined emulsion. Then 30 mL of HCl was added, and while rotated, the combined emulsion was heated up and kept at 80° C. for 6 hours. After completion of the polymerization reaction, obtained capsules were separated by filtration under reduced pressure, then washed with ion-exchanged water until the pH of the washing water was neutralized. The capsules were then dried for 5 days in a lyophilizer, thereby obtaining relatively bulky white capsules. Then the capsules were heat-treated at 150° C. for 5 hours. The yield was 95%. The average capsule diameter analyzed was 200 μm. The SEM observation showed that mononucleated voids were formed at the cross-section surface of the capsules.

### Example 29

Five grams of the void capsules having an inorganic wall obtained from Example 28 were dispersed in 50 mL of a saturated 1,3,5-trimethyl barbiturate (TMBA) solution, and shaken in a shaking apparatus at 23° C. for 1 week. Then these microcapsules enclosing the saturated TMBA solution were separated by filtration. After washed with a very small amount of ion-exchanged water, the microcapsules were dispersed in 50 mL of a 1 wt % ethylcellulose/dichloromethane solution. The suspension was then instantaneously dried using a spray drier with an inlet temperature of 80° C., a supply rate of 50 mL/min, a blowing volume of 0.5 m³/min, and a spraying pressure of 100 KPa in order to dry the outer shells, thereby obtaining microcapsules having an ethylcellulose outer coating, a silicon oxide inner shell, and an encapsulated substance of a saturated 1,3,5-trimethyl barbiturate solution.

### Examples 30-34

In place of the saturated TMBA solution used in Example 29, saturated solutions containing compounds listed in Table 7 were used for microencapsulation.

TABLE 7
Synthesis of capsules having an inorganic capsule wall enclosing various
encapsulated substances

| Example | Encapsulated substance |
|---|---|
| 30 | saturated N,N-dihydroxyethyl-p-toluidine solution (DEPT) |
| 31 | saturated p-sodium toluenesulfinate solution (p-TSNa) |
| 32 | t-butylhydroperoxide (TBHP) |
| 33 | saturated potassium persulfate solution |
| 34 | saturated ascorbate solution |

### Example 35

Five grams of the void capsules having an inorganic wall obtained from Example 28 were placed under a high vacuum of 0.05 Torr, and 50 mL of triethylene glycol dimetharylate (TEGDMA) was added under agitation using an electromagnetic agitator. Then these microcapsules enclosing the TEGDMA were separated by filtration. After washed with a very small amount of ethanol, the microcapsules were dispersed in 50 mL of a 1 wt % PVA solution. Its suspension was then instantaneously dried using a spray drier with an inlet temperature of 100° C., a supply rate of 50 mL/min, a blowing volume of 0.5 m³/min, and a spraying pressure of 100 KPa in order to dry the outer shells, thereby obtaining microcapsules having a polyvinyl alcohol outer coating, a silicon oxide inner shell, and an encapsulated substance of TEGDMA.

### Examples 36-40

In place of TEGOMA used in Example 29, compounds listed in Table 8 were used for microencapsulation.

TABLE 8
Synthesis of capsules having an inorganic capsule wall enclosing various
encapsulated substances

| Example | Encapsulated substance |
|---|---|
| 36 | Ethylene glycol dimethacrylate (EGDMA) |
| 37 | triethanol amine |
| 38 | gamma-methacryloxypropyltrimetoxy silane |
| 39 | 2-hydroxyethyl methacrylate (2-HEMA) |
| 40 | styrene |

### Example 41

### Microencapsulation of Polyacrylic Acid Solution by the Drying-in-Liquid Method

Materials used were glass ionomer cement CX-Plus liquid (manufactured by Shofu, Inc.) as a core material; condensed hexaglycerin recinoleate (Sunsoft 818SX: manufactured by Taiyo Kagaku Co., Ltd.) as a surfactant that forms an organic phase; polymethyl methacrylate (PMMA: manufactured by Mitsubishi Rayon Co., Ltd.) as a wall material polymer; ethyl acetate as an organic solvent; ion-exchanged water as outer phase water, and gelatin (manufactured by Nacalai Tesque, Inc.) as a dispersion stabilizer for outer phase water.

Firstly, 10 g of PMMA was dissolved in 50 mL of ethyl acetate containing 0.60 g of condensed hexaglycerin recinoleate. Then, 25 mL of CX-Plus liquid, i.e. an inner water phase of the core material, was stirred into the resultant organic phase for 10 minutes using a Physcotron homogenizer at 5000 rpm under cooling at 10° C. to obtain a primary dispersion phase. Next, gelatin was stirred and dissolved into 300 mL of ion-exchanged water at 60° C. to make of 3 wt %, so as to prepare an outer water phase. Then, while keeping the temperature of the outer water phase at 40° C., the primary dispersion phase was added using a three-one motor agitator at 500 rpm to obtain a combined emulsion. While rotated with the temperature maintained, the combined emulsion was agitated for 6 hours under slightly-reduced pressure. After desolvation of the emulsion, obtained microcapsules were separated by filtration under reduced pressure, then washed with copious ion-exchanged water, and dried with hot air at 40° C., thereby obtaining microcapsules enclosing pale yellow polyacrylic acid solution. The yield was 95%. The average capsule diameter analyzed was approximately 150 μm. The SEM observation showed that the structure of the capsules was relatively multinucleated.

### Example 42

### Microencapsulation of an Organic Powder Dispersive Monomer by the Drying-in-Liquid Method

Materials used were a combined monomer suspension of 60 wt % Bis-GMA and 40 wt % TEGDMA having 50 wt % glass ionomer cement CX-Plus powder (manufactured by Shofu, Inc.) dispersed as a core material; polyvinyl alcohol (PVA) as a wall material polymer; castor oil as outer phase oil; dehydrated ethanol as a diluent and flocculant; and formalin and a 6M hydrogen chloride solution as PVA curing agents.

Firstly, 25 mL of a combined monomer suspension of Bis-GMA/TEGDMA (60/40, wt %) having 50 wt % glass ionomer cement CX-Plus powder dispersed as a core material phase was stirred into 50 mL of a 10 wt % PVA solution for 10 minutes using a Physcotron homogenizer at 750 rpm under cooling at 10° C. to obtain a primary dispersion phase. Next, the primary dispersion phase was added to 300 mL of castor oil using a three-one motor agitator at 500 rpm under cooling at 10° C. to obtain an [(o/w)o] type combined emulsion.

Then, 300 mL of dehydrated ethanol was added, and the PVA solution phase, i.e. a wall material precursor, was partly dehydrated. Then 5 mL of 6M hydrogen chloride solution and 15 mL of formalin were added, and the mixture was heat-cured at 90° C. for 20 minutes. Then it was filtrated under reduced pressure, washed with copious ion-exchanged water, and dried in an oven at 40° C. to obtain microcapsules. The yield was 85%. The average capsule diameter analyzed was approximately 250 μm. The color of the capsules was slightly subdued beige.

### Example 43

### Microencapsulation by the Interfacial Polymerization Method

Firstly. 0.35 g of 1,6-hexanediamine (manufactured by Aldrich, Co.), 0.36 g of sodium carbonate and 0.5 g of TW-20 (manufactured by Dai-ichi Kogyo Seiyaku Co., Ltd.) were dissolved in 50 g of ion-exchanged water to obtain a water phase. Then, 25 g of triethylene glycol dimethacrylate (TEGDMA: manufactured by Shin-Nakamura Chemical Co., Ltd.) was stirred into the obtained water phase for 10 minutes using a Physcotron homogenizer at 700 rpm under cooling at 10° C. to obtain an [o/w] primary dispersion phase. Next, the primary dispersion phase was added to 300 mL of a combined organic solvent of chloroform/cyclohexane (=¼) containing 1 wt % of Solgen 30 (manufactured by Dai-ichi Kogyo Seiyaku Co., Ltd.) using a three-one motor agitator at 500 rpm under cooling at 5° C. to obtain an [(o/w)o] type combined emulsion. While the emulsion was still stirred, 50 mL of a combined organic solvent of chloroform/cyclohexane (=¼) having 0.55 g of adipic acid chloride (manufactured by Aldrich, Co.) dissolved was added. After approximately one hour of agitation, microcapsules were separated by filtration, and washed with cyclohexane, thereby obtaining microcapsules endosing TEGDMA. The yield was 97%. The average capsule diameter analyzed was approximately 70 μm. The color of the capsules was slightly white with high transparency.

### Examples 44-48

In place of TEGDMA used in Example 43, compounds listed in Table 9 were used for microencapsulation.

TABLE 9
Microencapsulation of monomers

| Example | Encapsulated substance |
|---|---|
| 44 | Ethylene glycol dimethacrylate (EGDMA) |
| 45 | Combined monomer of Bis-GMA/EGDMA (40/60, wt %) |
| 46 | Combined monomer of Bis-GMA/EGDMA (30/70, wt %) |
| 47 | Combined monomer of Bis-GMA/EGDMA (40/60, wt %) |
| 48 | Combined monomer of Bis-GMA/EGDMA (30/70, wt %) |

### Example 49

### Preparation of One-paste Glass Ionomer

Ten grams of 1 wt % polyvinyl alcohol, 5 g of the microcapsules obtained from Example 1 and 7 g of glass ionomer cement CX-Plus powder (manufactured by Shofu, Inc.) were placed in a 100 mL polyethylene vessel and kneaded for 5 minutes at 23° C. using a Non-bubbling Kneader NBK-2 (manufactured by Thinky Corporation) at 200 rpm to prepare a one-paste glass ionomer cement. The resultant one-paste glass ionomer cement was filled in an airtight container to conduct a forced test at 50° C. No gelation was observed in the cement even after 3 months. When 1 g of the one-paste glass ionomer cement was subjected to 200 W-20 kHz output of ultrasonic vibration for 10 seconds at 23° C., the cement was gelated at 3 minutes after the start of the vibration, and became completely hardened at 9 minutes.

### Examples 50-57

### Preparation of One-paste Glass Ionomer Cement

Basic formulation and procedure were the same as in Example 49, except for using 5 g of the various microcapsules enclosing CX-Plus liquid obtained from Examples 2 to 9 to prepare one-paste glass ionomer cement. The resultant one-paste glass ionomer cements were filled respectively in an airtight container to conduct a forced test at 50° C. In addition, 1 g of these one-paste glass ionomer cements were subjected to 200 W-20 kHz output of ultrasonic vibration for 10 seconds at 23° C. to measure the time required for gelation and curing. The test results are shown in Table 10.

TABLE 10
Curing time and life duration of one-paste glass ionomer cement

| | Example in | | | |
|---|---|---|---|---|
| | which added | Time required | Time required | |
| | capsules | for gelation | for curing | Condition after |
| Ex- | were | at 23° | at 23° | 3-month forced |
| ample | prepared | C. (min.) | C. (min.) | test at 50° C. |
| 50 | 2 | 3.0 | 9.0 | No gelation |
| 51 | 3 | 2.5 | 8.5 | No gelation |
| 52 | 4 | 2.5 | 8.0 | No gelation |
| 53 | 5 | 2.5 | 8.0 | No gelation |
| 54 | 6 | 2.0 | 7.5 | No gelation |
| 55 | 7 | 2.0 | 7.0 | No gelation |
| 56 | 8 | 1.5 | 6.5 | No gelation |
| 57 | 9 | 1.5 | 6.0 | No gelation |

### Example 58

### Preparation of One-paste Glass Ionomer Cement

Ten grams of 1 wt % polyvinyl alcohol, 5 g of the microcapsules obtained from Example 1, 1 g of the microcapsules obtained from Example 10, and 7 g of glass ionomer CX-Plus powder (manufactured by Shofu, Inc.) were placed in a 100 mL polyethylene vessel and kneaded for 5 minutes at 23° C. using a Non-bubbling Kneader NBK-2 (manufactured by Thinky Corporation) at 200 rpm to prepare a one-paste glass ionomer cement. The resultant one-paste glass ionomer cement was filled in an airtight container to conduct a forced test at 50° C. No gelation was observed in the cement even after 3 months. When 1 g of the one-paste glass ionomer cement was subjected to 200 W-20 kHz output of ultrasonic vibration for 10 seconds at 23° C., the cement was gelated at 3.5 minutes after the start of the vibration, and became completely hardened at 8 minutes.

### Examples 59-66

### Preparation of One-paste Glass Ionomer Cement

Basic formulation and procedure were the same as in Example 58, except for using the various microcapsules enclosing CX-plus liquid obtained from Examples 2 to 9, as well as the microcapsules endosing tartaric acid obtained from Examples 11 to 18 to prepare a one-paste glass ionomer cement.

The resultant one-paste glass ionomer cements were filled respectively in an airtight container to conduct a forced test at 50° C. In addition, 1 g of these one-paste glass ionomer cements were subjected to 200 W-20 kHz output of ultrasonic vibration for 10 23° C. to measure the time required for gelation and curing. The test results in Table 11.

TABLE 11
Curing time and life duration of one-paste glass ionomer cement

| | Example in | | | |
|---|---|---|---|---|
| | which added | Time required | Time required | |
| | capsules | for gelation | for curing | Condition after |
| Ex- | were | at 23° | at 23° | 3-month forced |
| ample | prepared | C. (min.) | C. (min.) | test at 50° C. |
| 59 | 2, 11 | 3.5 | 8.5 | No gelation |
| 60 | 3, 12 | 2.5 | 9.0 | No gelation |
| 61 | 4, 13 | 3.0 | 8.5 | No gelation |
| 62 | 5, 14 | 3.0 | 8.5 | No gelation |
| 63 | 6, 15 | 2.5 | 8.5 | No gelation |
| 64 | 7, 16 | 3.0 | 7.5 | No gelation |
| 65 | 8, 17 | 2.0 | 7.0 | No gelation |
| 66 | 9, 18 | 2.5 | 7.0 | No gelation |

### Example 67

### Preparation of One-paste Resin Modified Glass Ionomer Cement

Ten grams of a combined monomer of Bis-GMA(40 wt %)/TEGDMA(60 wt %). 7 g of the microcapsules enclosing a polyacrylic acid obtained from Example 19, 0.5 g of the microcapsules enclosing a catalyst (saturated potassium persulfate solution) obtained from Example 33, 0.5 g of the microcapsules enclosing a catalyst (saturated ascorbate solution) obtained from Example 34, and 7 g of glass ionomer CX-Plus powder (manufactured by Shofu, Inc.) were placed in a 100 mL polyethylene vessel and kneaded for 5 minutes at 23° C. using a Non-bubbling Kneader NBK-2 (manufactured by Thinky Corporation) at 200 rpm to prepare a one-paste resin modified glass ionomer cement.

The resultant one-paste glass ionomer cement was filled in an airtight container to conduct a forced test at 50° C. No gelation was observed in the cement even after 3 months. When 1 g of the one-paste resin modified glass ionomer cement was subjected to 200 W-20 kHz output of ultrasonic vibration for 10 seconds at 23° C., the cement was gelated at 3.5 minutes after the start of the vibration, and became completely hardened at 9 minutes.

### Examples 68-75

### Preparation of One-paste Resin Modified Glass Ionomer Cement

Basic formulation and procedure were the same as in Example 67, except for using the microcapsules enclosing glass ionomer CX-Plus liquid and hydroxycarboxylic acids obtained from Examples 20 to 27 to prepare a one-paste resin modified glass ionomer cement. The resultant one-paste glass ionomer cements were filled respectively in an airtight container to conduct a forced test at 50° C. In addition, 1 g of these one-paste resin modified glass ionomer cements were subjected to 200 W-20 kHz output of ultrasonic vibration for 10 seconds at 23° C. to measure the time required for gelation and curing. The test results are shown in Table 12.

TABLE 12
Curing time and shelf life of one-paste resin modified
glass ionomer cement

| | Example in | | | |
|---|---|---|---|---|
| | which added | Time required | Time required | |
| | capsules | for gelation | for curing | Condition after |
| Ex- | were | at 23° | at 23° | 3-month forced |
| ample | prepared | C. (min.) | C. (min.) | test at 50° C. |
| 68 | 20 | 3.5 | 8.0 | No gelation |
| 69 | 21 | 3.5 | 8.5 | No gelation |
| 70 | 22 | 3.0 | 8.5 | No gelation |
| 71 | 23 | 2.5 | 9.0 | No gelation |
| 72 | 24 | 3.5 | 8.5 | No gelation |
| 73 | 25 | 3.0 | 7.5 | No gelation |
| 74 | 26 | 2.0 | 8.0 | No gelation |
| 75 | 27 | 2.5 | 7.0 | No gelation |

### Example 76

### Preparation and Curing Time of One-paste Resin Modified Glass Ionomer Cement

Ten grams of glass ionomer CX-plus liquid (manufactured by Shofu, Inc.), 10 g of the microcapsules enclosing glass ionomer CX-Plus power and monomers obtained from Example 42, 0.5 g of the microcapsules enclosing a catalyst (saturated potassium persulfate solution) obtained from Example 33, and 0.5 g of the microcapsules endosing a catalyst (saturated ascorbate solution) obtained from Example 34 were placed in a 100 mL polyethylene vessel, and kneaded for 5 minutes at 23° C. using a Non-bubbling Kneader NBK-2 (manufactured by Thinky Corporation) at 200 rpm to prepare a one-paste resin modified glass ionomer cement. The resultant one-paste glass ionomer cement was filled in an airtight container to conduct a forced test at 50° C. No gelation was observed in the cement even after 3 months. When 1 g of the one-paste resin modified glass ionomer cement was subjected to 200 W-20 kHz output of ultrasonic vibration for 10 seconds at 23° C., the cement was gelated at 4 minutes after the start of the vibration, and became completely hardened at 10 minutes.

[Bond and Compressive Strengths of One-paste (Resin Modified) Glass Ionomer Cement at Enamel/Dentin-metal Interface]

Bond and compressive strengths of a one-liquid curable composition of the present invention at the enamel/dentin-metal interface were examined using the one-paste glass ionomer cements obtained from Examples 50-66 and the one-paste resin modified glass ionomer cements obtained from Examples 67-76.

[Shear Bond Test]

In place of human teeth, freshly extracted bovine anterior teeth were used. After cutting a dental root to remove a pulp, a bovine tooth was embedded in an epoxy resin. The labial side of the bovine enamel and dentin was polished under pouring water with the use of a No.80 water-proof abrasive paper, followed by a No.600 abrasive paper. The polished tooth surface was dried with oil-free compressed air. The adherend surface of a stainless rod (5 mm diameter, 12 mm length) was sandblasted with aluminium oxide powder. Next, 1 g of the one-paste (resin modified) glass ionomer cements obtained from Examples 50-76 were subjected to 200 W-20KHz output of ultrasonic vibration for 10 seconds at 23° C. to induce a reaction. Then the stainless rod was bonded to the aforesaid treated tooth surface mediated by each reacted paste, and subjected to a 200 g load. Excess paste pressed out of the bonding interface was removed using a blade. Then the test specimens were allowed to stand for 1 hour in a wet box at a humidity of 100%, and immersed in distilled water at 37° C. At 24 hours after bonding, a shear bond strength of each test specimen was measured using an Instron universal testing machine (Instron 5567, Instron Corporation) at a crosshead speed of 1 mm/min. to obtain a mean value of n=7. The results are shown in Table 13.

[Compressive Strength Measurement]

Test specimens (3 mm diameter, 6 mm height) were prepared for a compression test in accordance with the ISO standards using the one-paste (resin modified) glass ionomer cements obtained from Examples 50-76. The prepared specimens were immersed in distilled water at 37° C. for 24 hours. Then the compressive strength of each specimen was measured using an Instron universal testing machine at a crosshead speed of 1 mm/min. to obtain a mean value of n=5. The results are shown in Table 13.

TABLE 13
Bond and compressive strengths (MPa) of one-paste resin modified glass
ionomer cement at enamel/dentin-metal interface

| Example in which | Enamel-metal | Dentin-metal | Compressive |
|---|---|---|---|
| cement was | bond strength | bond strength | strength |
| prepared | (MPa) | (MPa) | (MPa) |
| 50 | 1.6 | 1.8 | 180 |
| 52 | 1.3 | 1.8 | 185 |
| 53 | 1.6 | 2.0 | 190 |
| 54 | 1.0 | 1.9 | 195 |
| 55 | 1.5 | 1.6 | 198 |
| 56 | 2.2 | 1.1 | 189 |
| 57 | 1.2 | 1.8 | 198 |
| 58 | 1.8 | 2.2 | 200 |
| 59 | 1.4 | 1.8 | 193 |
| 60 | 1.7 | 1.5 | 203 |
| 61 | 1.5 | 1.6 | 198 |
| 62 | 1.2 | 1.3 | 201 |
| 63 | 2.0 | 1.5 | 188 |
| 64 | 2.1 | 2.1 | 205 |
| 65 | 1.9 | 1.8 | 208 |
| 66 | 2.2 | 1.6 | 183 |
| 67 | 7.4 | 4.4 | 230 |
| 68 | 6.4 | 4.8 | 228 |
| 69 | 6.1 | 5.3 | 239 |
| 70 | 5.9 | 5.8 | 240 |
| 71 | 6.6 | 6.3 | 237 |
| 72 | 7.2 | 5.2 | 243 |
| 73 | 6.4 | 4.6 | 228 |
| 74 | 5.7 | 5.4 | 241 |
| 75 | 7.2 | 4.4 | 232 |
| 76 | 6.3 | 4.3 | 233 |

### Comparative Example 1

### Preparation of One-paste Resin Modified Glass Ionomer Cement

Ten grams glass ionomer CX-Plus liquid (manufactured by Shofu, Inc.), 20 g of glass ionomer CX-Plus powder, 5 g of 2-HEMA, 0.5 g of potassium persulfate, and 0.5 g of saturated ascorbate solution were placed in a 100 mL polyethylene vessel, and kneaded for 5 minutes at 23° C. using a Non-bubbling Kneader NBK-2 (manufactured by Thinky Corporation) at 200 rpm to prepare a one-paste resin-modified glass ionomer cement. However, increased viscosity was observed immediately after preparation, and gelation was fully induced after 15 minutes.

### Comparative Example 2

### Preparation of One-paste Glass Ionomer Cement

Ten grams of glass ionomer CX-Plus liquid (manufactured by Shofu, Inc.) and 20 g of glass ionomer CX-Plus powder were placed in a 100 mL polyethylene vessel and kneaded for 5 minutes at 23° C. using a Non-bubbling Kneader NBK-2 (manufactured by Thinky Corporation) at 200 rpm to prepare a one-paste glass ionomer cement. However, increased viscosity was observed immediately after preparation, and gelation was fully induced after 10 minutes.

As described above in Comparative examples where materials are not microencapsulated, not surprisingly, acid-base components initiate reaction and redox catalytic systems generate radicals, which suggests that they are not capable of coexisting with curable monomers. The present invention enables coexistence of these reactive components by microencapsulating such components, and also eliminates the cumbersome mixing procedure. These features will largely contribute to the medical and dental fields.

1. A one-liquid curable composition comprising micro- and/or nano-capsules.

2. The one-liquid curable composition of claim 1, wherein said micro- and/or nano-capsules have an encapsulated substance which is at least one material selected from the group consisting of radically-polymerizable monomers, polymerization initiators, polymerization accelerators, electrolyte polymers, electrolyte solutions, hydroxycarboxylic acids, hydroxycarboxylic acid solutions, acid-reactive filler, and acid-inactive fillers.

3. The one-liquid curable composition of claim 1, wherein in addition to said micro- and/or nano-capsules, at least one material selected from the group consisting of radically-polymerizable monomers, polymerization initiators, polymerization accelerators, electrolyte polymers, electrolyte polymer solutions, hydroxycarboxylic acids, hydroxycarboxylic acid solutions, acid-reactive fillers, and acid-inactive fillers can be arbitrarily added.
